(19) United States
(12) Patent Application Publication
(10) Pub. No.: US 2025/0291974 A1
(43) Pub. Date: Sep. 18, 2025

(54) INCREMENTAL TECHNIQUES FOR PRIVACY-PRESERVING DATA ANALYSIS

(71) Applicants: CASE WESTERN RESERVE UNIVERSITY, Cleveland, OH (US); Rutgers University, New Brunswick, NJ (US); UTHealth Houston, Houston, TX (US)

(72) Inventors: Erman Ayday, Cleveland, OH (US); Jaideep Vaidya, Cleveland, OH (US); Xiaoqian Jiang, Cleveland, OH (US)

(21) Appl. No.: 19/079,564

(22) Filed: Mar. 14, 2025

Related U.S. Application Data
(60) Provisional application No. 63/566,033, filed on Mar. 15, 2024.

(51) Int. Cl. G06F 30/20; G16B 20/20

(52) U.S. Cl. CPC G06F 30/20; G16B 20/20

(57) ABSTRACT

An example method can include generating first metadata representative of a first proper subset of data units in each of a plurality of samples stored in a first dataset, in which each sample of the plurality of samples stored in the first dataset includes a plurality of data units. The method can also include generating second metadata representative of a second proper subset of data units in each of a plurality of samples stored in a second dataset, in which each sample of the plurality of samples stored in the second dataset includes a plurality of data units. The method can also include sending the first and second metadata to a computer. The method can also include calculating a measure of relatedness between the samples stored in the first dataset and the samples stored in the second dataset based on the first metadata and the second metadata.

### RELATED APPLICATION

This application claims priority from U.S. Provisional Application No. 63/566,033, filed Mar. 15, 2024, the subject matter of which is incorporated herein by reference in its entirety.

### GOVERNMENT FUNDING

This invention was made with government support under LM013429 and LM014520, awarded by the National Institutes of Health. The government has certain rights in the invention.

### TECHNICAL FIELD

This disclosure relates to systems and methods of preserving privacy across datasets, such as when determining relatedness of samples in the datasets.

### BACKGROUND

When various types of data are shared between two or more entities, there may be a need to preserve the privacy the data being shared, such as to conceal or obfuscate the identity or source of such data. For example, the use of genomic data in collaborative studies can have privacy implications, as it includes information about a subject's phenotype, ethnicity, family memberships, and disease conditions, which might be highly sensitive to the study participants. One often overlooked step in privacy-preserving collaborative studies is the identification of records (samples) that are to be included and/or filtered for the collaborative study. The identification of such records (samples) is fundamental to ensure that the results of the collaborative study are based on high quality data.

### SUMMARY

This disclosure relates to systems and methods of preserving privacy across datasets, such as for determining relatedness of samples in the datasets.

A described example relates to a method. The method can include generating, on or by a first computer, first metadata representative of a first proper subset of data units in each of a plurality of samples stored in a first dataset, in which each sample of the plurality of samples stored in the first dataset includes a plurality of data units. The method can also include generating, on or by a second computer, second metadata representative of a second proper subset of data units in each of a plurality of samples stored in a second dataset, in which each sample of the plurality of samples stored in the second dataset includes a plurality of data units. The method can also include sending the first metadata to a third computer through a first communications link. The method can also include sending the second metadata to the third computer through a second communications link. The method can also include calculating, by the third computer, a measure of relatedness between the samples stored in the first dataset and the samples stored in the second dataset based on the first metadata and the second metadata. The method can also include sending the measure of relatedness to each of the first computer and the second computer.

Another described example includes a method. The method includes selecting a proper subset of data units in each of a plurality of samples stored in a first dataset, in which each sample of the plurality of samples stored in the first dataset includes a respective plurality of data units. The method can also include generating metadata based on the selected subset of data units. The method can also include sending the metadata to a second computer through a communications link. The method can also include receiving a measure of relatedness determined by the second computer, in which the measure of relatedness is based on the generated metadata and other metadata from at least one other computer, and the measure of relatedness quantifies a similarity between samples based on the proper subset of data units for samples stored in the first dataset and a proper subset of data units for samples stored in at least one other dataset. The method can also include updating the first dataset based on the received measure of relatedness, in which each of the selecting, generating, sending, receiving and updating is repeated for a plurality of iterations, and the updating performed for at least one iteration is based on measures of relatedness for a current iteration and one or more preceding iterations.

Another described example provides a system that includes non-transitory memory and one or more processors. The memory can store instructions and data, in which the data comprises a first dataset that includes a plurality of samples, each sample including a plurality of data units. The one or more processors are coupled to the memory, in which the instructions are executable by the one or more processors. The instructions can include synchronization code to select a proper subset of the data units in each of a plurality of samples stored in the first dataset, in which each sample of the plurality of samples stored in the first dataset includes a respective plurality of data units. The instructions can include metadata generator code to generate metadata based on the selected subset of data units. The instructions can include outsource code to send the generated metadata to a server computer through a communications link. The outsource code can also to receive a measure of relatedness from the server computer. The received measure of relatedness can be based on the generated metadata and other metadata from at least one other computer. The measure of relatedness can quantify a similarity between samples based on the proper subset of data units for samples stored in the first dataset and a proper subset of data units for samples stored in at least one other dataset. The instructions can include update code to update the first dataset based on the received measure of relatedness, in which each of the synchronization code, the metadata generator code, the outsource code, and the update code is executed for each of a plurality of iterations, and the updating at each of the iterations is based on measures of relatedness for a current iteration and one or more prior iterations.

### BRIEF DESCRIPTION OF THE DRAWINGS

FIG. 1 depicts an example of a system to enable privacy preservation within a shared framework.

FIG. 2 depicts an example of a synchronization module of a privacy preserving system.

FIG. 3 depicts an example of an initialization module of a privacy preserving system.

FIG. 4 depicts an example of an update module of a privacy preserving system.

FIG. 5 depicts a workflow diagram for an example privacy preserving system.

FIG. 6 depicts an example of pseudo code that can be implemented in a privacy preserving system.

FIG. 7 depicts examples of graphs plotting accuracy for different degrees of relatedness as a function of the number of shared data units, which can be determined by systems and methods described herein.

FIG. 8 is a flow diagram depicting an example method to preserve privacy for collaborative data analysis.

### DETAILED DESCRIPTION

This disclosure relates to systems and methods to preserve privacy across multiple datasets for collaborative data analysis, which can be implemented within a shared computing framework (e.g., a federated data system). For example, the datasets can be controlled by separate independent entities (also referred to herein, depending on context, as computers, clients, or researchers) operating in the shared computing framework for analyzing samples (e.g., data records) stored in the respective datasets, such as for identifying a relatedness of samples in the respective datasets. As used herein, relatedness can refer to an amount of similarity or dissimilarity among data units. As a further example, in the context of genomic studies (e.g., genomic testing and genome-wide association studies (GWAS)), the relatedness can describe or quantify a kinship relationship among genetic information represented in sample records. While many examples described herein are in the context of genetic information and analysis, in other examples, the systems and methods for privacy preserving and data analysis can be implemented to preserve privacy and characterize similarities in other contexts and for other types of information.

As an example, a computer-implemented method can be used by two or more entities operating in a shared framework (e.g., a federated data system). For examples, each of the entities can be individuals (e.g., researchers or other data analysists), computers being used by or under the control the respective individuals, or both the individuals and their respective computers. As described herein, the term computer can refer to any device or combination of devices including a processor and non-transitory memory to store data and executable instructions. In the shared framework, each entity can operate independently and include a respective dataset that includes a set of samples (e.g., data records), such as corresponding to samples of genetic information of subjects, each having a respective plurality of data units (e.g., single nucleotide polymorphisms (SNPs)). Each entity can select a proper subset of data units for a plurality of samples stored in a dataset, which is stored in or accessible by the respective entity. The selected subset of data units can be synchronized among the entities, such as corresponding to an agreed upon fraction of the data units (e.g., a common fraction of SNPs). The synchronization between entities can be based on communication between the entities, which can be implemented through the shared framework and/or outside of the shared framework. Also, or as an alternative, the number of data units in the selected subsets can be the same or different for each iteration.

Each of the entities can be configured to generate metadata to describe samples in the respective entity's set of samples based on the selected proper subset of data units. The metadata for a given entity can represent the proper subset of the data units for the given entity's sample. By generating metadata for a subset of the samples, as described herein, the identity of the samples and/or the source of such samples can be obfuscated. Each entity can send the metadata to a shared resource (e.g., a shared computer, such as a server) through a communications link. The shared resource is configured to calculate a pairwise measure of relatedness of the samples of each of the entities based on the metadata provided by the respective entities. The measure of relatedness can quantify a similarity between the proper subset of data units for samples stored datasets of each of the entities (e.g., coefficients or other values representing kinship or another genetic relationship). The shared resource can return the calculated pairwise measure of relatedness to the respective entities. Each of the entities can in turn update the plurality of samples stored in the dataset based on the measure of relatedness. The computer implemented method can be an iterative process, in which each of the selecting, generating, sending, receiving and updating is repeated for a plurality of iterations. The resulting updated measures of relatedness in the respective datasets can be used to control further data analysis of the information in the datasets. For example, the updated measures of relatedness can be used to remove or retain a relevant subset of samples from the samples stored in a given dataset, and the analysis can be performed with respect based on the modified sample set. For example, samples having a certain degree of kinship can be retained or removed for genomic testing or analysis between federated datasets.

The systems and methods described herein can be configured to implement what is referred to as an incremental update kinship identification framework (referred to herein as an INK framework), in which all the entities (e.g., researchers, data analysts) generate the metadata and monitor the kinship relationships of each pair of subjects in the federated setting iteratively. Also, or alternatively, the entities can make use of auxiliary information (e.g., simulation distributions of relatedness coefficients) to determine cutoff values for classifying subjects belonging to different categories of relatedness (e.g., kinship categories). Also, or alternatively, the systems and methods can employ an informative score (IS) of each data unit (e.g., SNP), which can be defined based on the impact it has on the relatedness coefficients, and the data units (e.g., SNPs) can be selected for inclusion in subsets of data units based on according to a ranking of the informative scores.

Compared to other approaches, the systems and methods described herein can achieve higher kinship identification correctness with fewer data units (e.g., SNPs) to be shared with the server, thus reducing the privacy risk and enabling more efficient computations to be performed. To further enhance preserving privacy, the metadata sent for a current iteration can be unliked from metadata that is sent for other iterations. Also, each entity can perform updating for a current iteration based on measures of relatedness for the current iteration in combination and the measures of relatedness for any one or more preceding iterations. Thus, the systems and methods described herein can compute kinship relationships between samples in federated datasets with increased computational efficiency and with lower privacy risks than many existing approaches.

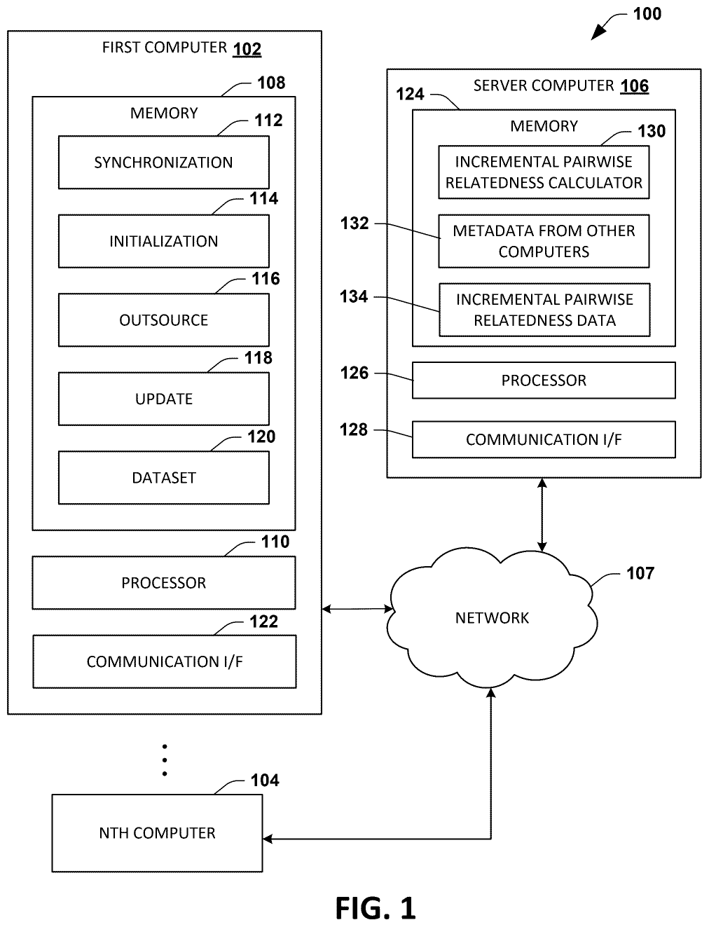

FIG. 1 depicts an example of a system 100 to enable privacy preservation within a shared computing framework, such as a federated system. The system 100 includes a number of computers 102 and 104, shown as the first computer and the Nth computer, where N is a positive integer greater than one denoting the number of computers. Each of the N computers 102, 104 can be associated with (or controlled by) one or more users, such as researchers or other data analysts. The system 100 also includes a server computer 106 (also referred to herein as a server). The server 106 can be implemented or controlled by a third party (e.g., a shared computing resource) that has sufficient computation power to enable collaborative data analysis and studies based on data shared by two more users (e.g., via computers 102-, 104). Each of the computers 102, 104, and 106 can be coupled to each other through one or more communication links, shown as including a network 107. The network 107 can include hardware, software, and/or firmware to enable communications of data between any or all of the computers 102, 106, 106 through one or more physical (e.g., wired or optical) and/or wireless communication links.

As an example, the first computer 102 can include memory 108, which can include one or more non-transitory machine-readable media to store data and executable instructions (e.g., program code). The computer 102 can also include one or more processors 110, each of which can include one or more processing cores, to access the memory 108 and execute corresponding instructions. In the example of FIG. 1, the instructions in the memory 108 include program code (e.g., methods or functions) including a synchronization module 112, an initialization module 114, an outsource module 116, and an update module 118. The memory 108 also includes a dataset 120, which can include a multitude of data samples, which can be processed and analyzed based on execution of the modules 112, 114, 116, and 118. The memory 108 and/or modules 112, 114, 116, and 118 can reside locally on the computer 102, be implemented remotely through a set on another computer (e.g., the server 106 or a cloud-based computing system), or be implemented in a distributed computing architecture that includes the computer 102 and one or more remote computers. The dataset 120 in each of the N computers 102, 104 can be separate and independent data, which can be of a confidential and/or private nature, such as genomic information for a plurality of subjects. As used herein, a subject can refer to any individual (e.g., human or other animal) for which associated data has been acquired and stored in a corresponding dataset. The dataset 120 can store the samples according to various types of data structures (e.g., linked lists, records, arrays, hash tables, trees, and the like), which can vary depending on the type of data. For example, the dataset 120 includes a plurality of data samples for respective subjects, in which each sample includes a plurality of data units (e.g., SNPs) for each of the subjects.

The first computer 102 can also include one or more communication interface 122 configured to enable communication between the first computer and the server computer 106 as well as, in certain examples, between the first computer and any of the N−1 computers 104. For example, the communications interface 122 can include a wireless communications network device configured to communicate data through a wireless network (e.g., network 107), such as a Wi-Fi, Bluetooth, or a cellular data link. Also, or as an alternative, the communications interface 122 can include a physical communications network device configured to communicate data through a wired or optical network (e.g., network 107), such as Ethernet, fiber channel, or the like. Also, or as an alternative, the communications interface 122 can be configured to implement secure connection (e.g., encrypted data communications) through the network 107.

The server computer 106 includes memory 124, which can include one or more non-transitory machine-readable media, to store data and executable instructions (e.g., program code). The server computer 106 can also include one or more processors 126, each of which can include one or more processing cores, to access the memory 124 and execute corresponding instructions, which can be based on data received from one or more of the computers 102, 104. The server computer 106 also includes one or more communication interfaces 128 configured to enable communication with the first computer 102 and one or more other computers 104.

In the example of FIG. 1, the memory 124 can store data received from one or more other computers 102, 104, including metadata 132 from such other computers. The memory 124 can also the instructions in the memory 108 include program code (e.g., modules) including an incremental pairwise relatedness calculator 130, which can be configured to compute incremental pairwise relatedness data 134 based on the metadata 132. As described herein, for example, the incremental pairwise relatedness data 134 can include coefficients (or other information) describing a kinship relationship between each pair of the data units represented by the metadata 132 from the N computers 102, 104.

For sake of brevity details of each of the other N−1 computers 104 have been omitted. In the example system 100 of FIG. 1, it is to be understood that each of the other of the N−1 computers 104 can be configured in a similar manner to the first computer 102 (e.g., including executable instructions and a dataset) to enable interoperation in the shared computing framework provided by the system 100. Different computing architectures can be used to implement any one or more of the computers 102, 104, 106 in other examples. As described herein, instead of outsourcing information all at once, each user (e.g., researcher or data analyst) breaks down the metadata and outsources a small subset of metadata for each of a plurality of iterations. Each of the modules 112, 114, 116, and 118 can be executed (e.g., by processor 110) at different parts (e.g., stages) of each iteration of the iterative process described herein.

By way of example, the synchronization module (e.g., instructions executable by the processor 110) 112 is configured to enable collaboration between users (at respective computers 102, 104) and define a proper subset of data units for samples (e.g., records) stored in the dataset 120. In examples herein, each of the data units can be an entry in a data record representing a genetic marker, such as an SNP, for a given subject. The proper subset of data units defined by the synchronization module 112 thus can represent a fraction of the SNPs in the samples stored in the dataset 120, which can be synchronized with a subset of data units being selected at one or more other computers 104. The synchronization module 112 can also authorize and/or enable collaboration between the respective users at the server computer 106. For example, users of each of two or more computers 102, 104 can communicate information through the communication link represented as including network 107 responsive to respective user inputs for determining a common fractional set of available data units in the respective datasets 120 of each computer 102-104.

As described herein, the relatedness of samples between two or more datasets can be determined over an iterative process based on a respective incremental subset of data units used at each iteration. Accordingly, the synchronization module 112 can be executed by the processor 110 for each of a plurality of iterations to identify a respective subset of the data units that is to be used for each respective iteration. The number of data units (a fraction of the total number of data units) can be the same for each iteration. Alternatively, the number of data units (a fraction of the total number of data units) can vary for each iteration. Also, or alternatively, the selection of data units for a respective iteration can be random or, in other examples, the data units can be selected (e.g., by the synchronization module) from among the data units to provide a greater impact on the measure of relatedness that is to be determined by the server. Each of the computers 102, 104 thus agree on which proper subset of data units to include at each iteration, and the selection and agreement on data units can be responsive to a user input at each computer by users who are collaborating. Alternatively, the selection and agreement on data units can be determined automatically or semiautomatically (e.g., a combination of manual and automated means). In some examples, the number of iterations can be a collaboration parameter that is set by the synchronization module 112 responsive to a user input by one or more users of computers 102, 104. In other examples, the number of iterations can be set (or determined) on the fly to ascertain a degree of relatedness (e.g., degree of kinship) to a desired level of confidence.

In some examples, the communication relating to synchronization of data units further can be provided through the communications interface 128 of the server 106. Also, or as an alternative, all or a portion of the synchronization of the selected subset of data can be communicated between the computers 102 and 104 through other communications channels, which may involve or not involve communicating through the server 106. In some examples, the synchronization module 112 can be configured (e.g., include executable instructions) to guide selection of the subset of data units by identifying which data units are expected to have a greater impact on the measure of relatedness that is to be determined (e.g., by calculating an impact score). The synchronization module 112 can be further configured to extract, tag, or otherwise identify the proper subset of selected data units from the dataset 120 for each computer 102, 104 that is collaborating.

The initialization module (e.g., instructions executable by the processor 110) 114 is configured to generate metadata representative of (e.g., characterizing) the proper subset of data units for the samples (or at least a portion thereof) stored in the dataset 120. Each individual that is collaborating through the server computer 106 can locally generate (by respective computer 102, 104) metadata for the same selected proper subset of data units (e.g., as provided by the synchronization module). The metadata can include identifiers for the selected proper subset of respective data units (e.g., as specified by the synchronization module) for the samples stored in the dataset 120. The identifiers can be encoded identifiers, in which each of encoded identifiers represents a respective data unit. For example, where each of the data units represents a genetic marker (e.g., an SNP), each of the encoded identifiers has a unique value that represents a respective genetic marker (e.g., respective SNP) without including the SNP IDs. Since the metadata does not contain the SNP IDs, the server cannot simply construct the SNP sequence of the samples that are included in the metadata.

By way of example, assume the original dataset 120 of one of the individual collaborators includes X records (people) having Y SNPs for each person and assume each record is in one row of the dataset and SNPs are in the columns. The initialization module 114 is programmed to generate the metadata for one iteration based on the selected proper subset of SNPs that has been determined in synchronization with one or more other collaborators. The columns for the selected SNPs can be reordered (e.g., shuffled responsive to a user input-manually, semiautomatically, or automatically) to introduce some obfuscation. Additionally, or alternatively, responsive to a user input instruction, the initialization module 114 can introduce some noise (e.g., random perturbations) to the selected and shuffled columns of SNPs to further obfuscate the real values of the SNPs. The resulting proper subset of SNP data can be stored in the memory 108 for subsequent processing, which can include generating metadata representative of the proper subset of SNPs.

The outsource module 116 (e.g., instructions executable by the processor 110) is configured to send the metadata to the server computer 106 through a corresponding communications link, including network 107. For example, the outsource module 116 can instruct the communication interface 122 to send the metadata over a secure communications link to the server computer 106. The server computer 106 can receive the metadata through its communication interface 128 and store the metadata from two mor more collaborating computers 102, 104 in memory 124, shown at 132, for processing.

The incremental pairwise relatedness calculator 130 (e.g., instructions executable by the processor 126) is configured to compute a measure of pairwise relatedness between the samples stored in dataset 120 of the computer and samples in one or more seconds dataset based on the metadata 132 received from respective computers for the proper subset of data units (e.g., incremental data). The incremental pairwise relatedness calculator 130 can implement one or more metrics to measure relatedness between samples based on the incremental metadata that is provided in each iteration. As described herein, the metadata received at the server at each iteration is unlinked (e.g., decoupled) from metadata at each other iteration to help preserve privacy. For example, incremental pairwise relatedness calculator 130 implements a separate and independent relatedness calculation based on the metadata received for iteration. That is, the server 106 receives no information to link metadata between different iterations and thus remains unaware of the iterative nature of the process, which is being implemented based on the instructions 112-118 executed at the respective computers 102, 104. Examples of some relatedness metrics that the incremental pairwise relatedness calculator 130 can compute include the kinship-based inference for GWAS (KING), the identity by descent estimation method, graphical representation of relationship errors, and kinship inference based on distance (KIND) to name a few.

As a further example, assuming that SNPs are biallelic, the incremental pairwise relatedness calculator 130 computes a KING kinship coefficient ϕ_{i,j} between a pair of samples i and j as follows:

$${ϕ}_{i , j} = \frac{2 {n}_{11} - 4 ( {n}_{02} + {n}_{20} ) - {n}_{* 1} + {n}_{1 *}}{4 {n}_{1 *}} , \qquad ( Eq . 1 )$$

where:
   n₁₁ represents the number of SNPs in which both samples are heterozygous,
   nₒ₂ in which sample i is homozygous dominant and sample j is homozygous recessive,
   n₂₀ refers to the opposite case (i.e., i is homozygous recessive and j is homozygous dominant), and
   n₁₊ and n₊₁ in which samples i and j are heterozygous, respectively.

The incremental pairwise relatedness calculator 130 can be configured to compute KING coefficients between each pair of SNP samples provided in the metadata 132 received from respective computers 102, 104. Based on KING, implemented by the incremental pairwise relatedness calculator 130, a kinship coefficient greater than different thresholds can imply different degrees of kinship relatedness. The server computer 106 is further configured to return the computed measure of relatedness between the samples to each of the collaborating computers 102, 104 that provided the metadata for a respective iteration.

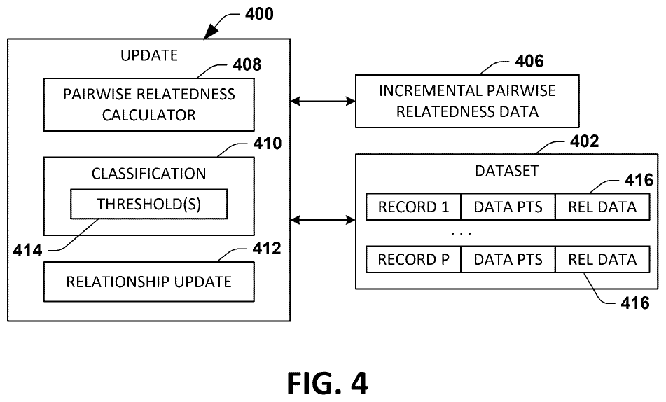

The computer 102, 104 for each collaborator can receive the computed measures of relatedness (e.g., pairwise relatedness data 134, such as KING coefficients) from the server 106 via the network 107 and store the relatedness data in the computer's memory 108 for further processing by the update module 118. The update module 118 (e.g., instructions executable by the processor 110) is configured to update the plurality of samples stored in the dataset based on the computed pairwise measure of relatedness for pairs of data units determined by the server 106 for a given iteration. The update module 118 can perform updating based on incremental relatedness data received from the server for multiple iterations. That is, unlike the relatedness computations performed at the server 106 by the incremental pairwise relatedness calculator 130, which only determines relatedness for metadata describing a respective subset of data units selected for a given (e.g., current) iteration, the update module 118 can take into account the incremental measure of relatedness computed for the given iteration as well as the measure of relatedness computed one or more preceding iterations, such as described herein (see, e.g., FIG. 4). As a result of iteratively outsourcing fractions of metadata describing the data units, each researcher is able to recover the results as computed on the whole metadata (or a larger subset of metadata) over a number of iterations based on the results returned at each iteration.

Continuing with the example where the computed measure of relatedness includes KING coefficients determined by the server for SNP pairs in the selected subset of SNPs, the update module 118 can compute a value for the relatedness based on the KING coefficients for each pair of the SNPs. The update module 118 can also classify a degree of the kinship relationship based on the computed relatedness value for each pair of the SNPs based on one or more thresholds. The updated relatedness information can be stored in the dataset 120 to specify the measure of relatedness for pairs of data units (e.g., SNPs) that have been included in one or more iterations.

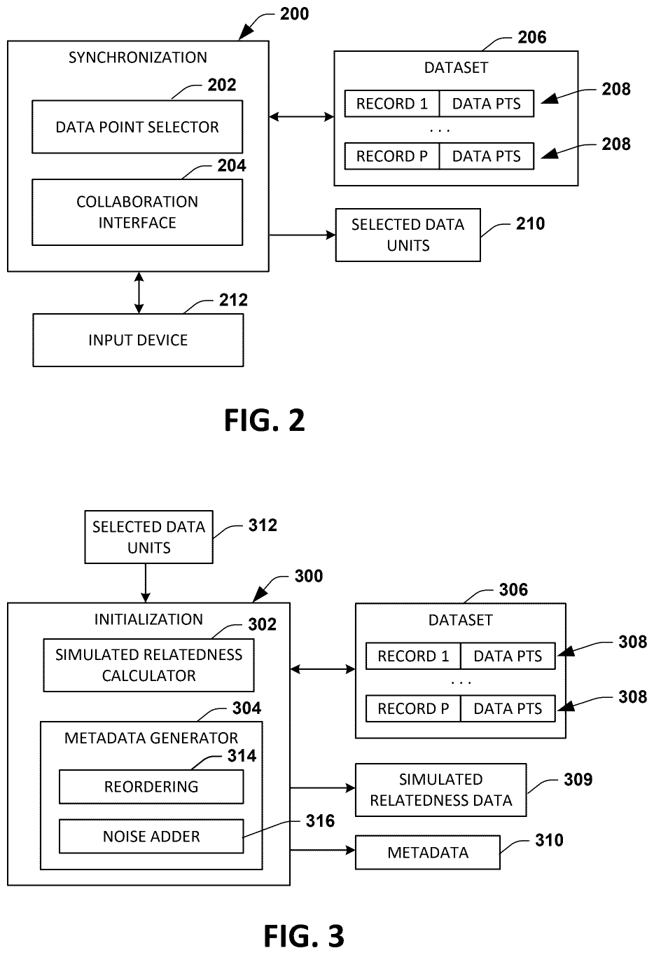

FIG. 2 depicts an example of a synchronization module 200 that can be implemented as part of a privacy preserving system (e.g., the system 100 of FIG. 1). The synchronization module 200 is a useful example of the synchronization module 112 of FIG. 1. Accordingly, the description of FIG. 2 can also refer to certain aspects of FIG. 1. As shown in FIG. 2, the synchronization module 200 includes a data unit selector 202 and a collaboration interface 204. The data unit selector 202 is configured to select a proper subset of the data units for records 208 stored in a dataset 206. In the example of FIG. 2, the dataset 206 includes P records 208, where P is a positive integer denoting the number of samples. Each of the records 208 can represent a sample of data for a subject, such as genomic data, in which each sample of genomic data includes a number of data units such as SNPs. The SNPs may reside within coding sequences of genes, non-coding regions of genes, or in an intergenic regions (e.g., regions between genes). Other types of data ca be used as data units in the records 208 in other examples.

As described herein, the synchronization module 200 enables synchronization of data units between collaborators for further analysis. Accordingly, the collaboration interface 204 is configured to enable collaboration between users (e.g., at respective computers 102, 104). The collaboration interface 204 can control (e.g., instruct) the data unit selector 202 to define a proper subset of data units, shown as selected data units 210, for the records 208 stored in the dataset 206. As described herein, each instance of the selected data units 210 that is generated can be stored in memory (e.g., memory 108) and used for determining relatedness of data units among samples in different datasets (e.g., federated datasets) for a respective iteration.

In some examples, a user can interact with the collaboration interface (e.g., a graphical user interface) 204 and/or the data unit selector 202 through one or more input devices 212 (e.g., a keyboard, a mouse, a touchscreen, a gesture control device, etc.), which can be part of or external to the computer 102, 104. A user can use the input device 212 to send messages to or otherwise communicate bi-directionally with one or more other user-collaborators as part of the synchronization of data units. A user can also interact with the synchronization module 200 to enable (or authorize) the collaboration to continue or to terminate the collaboration with one or more other users responsive to a user input instruction entered by the input device 212. For example, if the relatedness data for all the data samples does not change (e.g., converges) for two or more sequential iterations or for any other reason, one or more of the users can decide to terminate the collaboration responsive to a user input entered at the input device 212. In one example, the collaborating users can agree on and generate the selected data units 210 separately for each iteration, which can allow the users to evaluate updated relatedness information with each iteration. In another example, the collaborating users can agree on and generate multiple instances of the selected data units 210 to be used for each of a plurality of different iterations.

FIG. 3 depicts an example of an initialization module 300 that can be implemented as part of a privacy preserving system (e.g., the system 100 of FIG. 1). The initialization module 300 is a useful example of the initialization module 114 of FIG. 1. Accordingly, the description of FIG. 3 can also refer to certain aspects of FIG. 1. As shown in FIG. 2, the initialization module 300 includes a simulated relatedness calculator 302 and a metadata generator 304. Similar to the example of FIG. 2, the initialization module 300 can access a dataset 306 in memory that includes P records 308, where P is a positive integer denoting the number of records. Each of the records 308 can represent samples of data for a number of subjects, such as including SNPs as described herein.

As an example, the simulated relatedness calculator 302 (e.g., instructions executable by processor 110) is configured to generate simulated relatedness data 309 which can define one or more thresholds for categories of relatedness (e.g., relationship groups). For example, the simulated relatedness calculator 302 can generate a simulated set of data units for an artificial relatives and unrelated subjects (i.e., a simulated set of data units) following Mendel's Laws The simulated set of data units can contain artificially generated relatives (e.g., up to third-degree or higher degrees of relatives) for synthetic subjects unrelated to subjects in the dataset 306. The simulated relatedness calculator 302 can further calculate measures of relatedness (e.g., KING coefficients or other relatedness metrics), such as according to Eq. 1, on the simulated set of data units. The simulated relatedness calculator 302 can then determine aggregate statistics for each relationship group, which can be provided as the simulated relatedness data 309. The statistics can provide descriptive statistics (e.g., minimal, maximal, and mean) of the corresponding coefficient distributions to represent cutoff values (e.g., thresholds) for each relationship group (e.g., degree of kinship). In the example of FIG. 3, the simulated relatedness calculator 302 is shown as part of the initialization module 300; however, in other examples, the simulated relatedness calculator 302 can be separate from the initialization module 300 and/or be implemented as part of the synchronization module 112, 200.

As a further example, the metadata generator 304 is configured to generate metadata 310 based on selected data units 312 of the dataset (e.g., selected by synchronization module 112, 200 based on agreement between users). In the example of FIG. 3, the metadata generator includes a reordering function 314 and a noise adder function 316. The reordering function 314 (e.g., instructions executable by processor 110) is configured to reorder selected data units 312 of the dataset 306. For example, where the data units are SNPs organized as columns in a data structure representing the dataset 306, the reordering function 314 can reorder (e.g., shuffle) the columns for each of the selected SNPs to different columns in the dataset 306. The reordering of columns in the dataset 306 can be responsive to a user input selection, involve random or pseudorandom reordering, or a combination of manual and automated reordering. Additionally, or alternatively, the noise adder function 316 (e.g., instructions executable by processor 110) can be configured to introduce noise (e.g., random or fixed data perturbations) to the SNPs in each of selected and reordered columns of SNPs to obfuscate the real values of the SNPs. The metadata generator 304 can provide and store the metadata 310 representative of the selected data units (a fractional set of SNPs), which have been reordered and perturbed to introduce noise.

FIG. 4 depicts an example of an update module 400 that can be implemented as part of a privacy preserving system (e.g., the system 100 of FIG. 1). The update module 400 is a useful example of the update module 118 of FIG. 1. Accordingly, the description of FIG. 4 can also refer to certain aspects of FIG. 1. The update module 400 is configured to update relatedness information for a dataset 402, which includes data units in a plurality of records 404, based on incremental pairwise relatedness data 406. As described herein, the updating of relatedness information by the update module 400 at each of the iterations can be based on incremental pairwise relatedness data 406 received for the current iteration and any one or more prior iterations. In the example of FIG. 4, the update module 400 includes a pairwise relatedness calculator 408, a classification function 410, and a relationship update engine 412. The pairwise relatedness calculator 408 (e.g., instructions executable by processor 110) is configured to compute a measure of relatedness for corresponding data units in respective records 404 of the dataset 402 based on the incremental pairwise relatedness data 406 (e.g., received from the server 106 based on pooled metadata). The classification function 410 (e.g., instructions executable by processor 110) can be configured to classify or categorize a degree of relatedness (e.g., kinship degree) for respective pairs of data units based on the measure of relatedness computed by the calculator 408 for each pair of the data units relative one or more thresholds (e.g., thresholds defining degrees of relatedness, such as determined by initialization module 114, 300). The relationship update engine 412 (e.g., instructions executable by processor 110) is configured to update the dataset 402 to specify relatedness data 416 describing a measure of relatedness and/or degree of relatedness between a subset of data units in the records 404 and corresponding samples of another user. The relatedness data 416 can be appended to each record 404.

As an example, the data units are SNPs and the incremental pairwise relatedness data 406 from the server includes coefficients (e.g., KING coefficients) representing kinship relationships between respective pairs of samples represented by the pooled metadata for a fractional subset of SNPs (e.g., from computers 102, 104 of collaborating users). Other metrics can be used in other examples to measure the relatedness between samples. The pairwise relatedness calculator 408 can calculate the KING coefficients for sample pairs based on all the shared SNPs via Eq 2 below. As a further example, assume that at respective iterations t and t+p, users (e.g., researchers or data analysts) share metadata M^{t} and M^{t+p} to the server 106. Note that each metadata includes a small subset of SNPs, denoted as S^{t} and S^{t+p} respectively, and S^{t}∩S^{t+p}=∅. For example, given approximately 1000 SNPs, the subset of SNPs included in the metadata can be 50 SNPs, 100 SNPs, 200 SNPs, 300 SNPs or other fractional sets of the total number of SNPs. The incremental pairwise relatedness data 406 can include the KING coefficients computed on S^{t} and S^{t+p} to both researchers. Denote ϕ, ϕ^{t}, and ϕ^{t+p} as the KING coefficients computed on S^{t}∪S^{t+p}, S^{t}, and S^{t+p}, respectively. Then, the pairwise relatedness calculator 408 (e.g., executed by or on behalf of each of the researchers) can compute ϕ as follows:

$$ϕ = \frac{{n}_{1 *}^{t} {ϕ}^{t} + {n}_{1 *}^{t + p} {ϕ}^{t + p}}{{n}_{1 *}^{t} + {n}_{1 *}^{t + p}} \qquad ( Eq . 2 )$$

Note that this is true since it can be shown that:

$$\begin{matrix}ϕ = \frac{2 {n}_{11} - 4 ( {n}_{02} + {n}_{20} ) - {n}_{* 1} + {n}_{1 *}}{4 {n}_{1 *}} \\ = \frac{\begin{matrix}2 ( {n}_{11}^{t} + {n}_{11}^{t + p} ) - 4 ( ( {n}_{02}^{t} + {n}_{02}^{t + p} ) + ( {n}_{20}^{i} + {n}_{20}^{t + p} ) ) - \\ ( {n}_{* 1}^{i} + {n}_{* 1}^{t + p} ) + ( {n}_{1 *}^{t} + {n}_{1 *}^{t + p} )\end{matrix}}{4 ( {n}_{1 *}^{t} + {n}_{1 *}^{t + p} )} \\ = \frac{{n}_{1 *}^{t} {ϕ}^{t} + {n}_{1 *}^{t + p} {ϕ}^{t + p}}{( {n}_{1 *}^{t} + {n}_{1 *}^{t + p} )}\end{matrix} \qquad ( Eq . 3 )$$

The classification function 410 can be configured to compare the results with the aggregate statistics and obtain a kinship relationship for each pair of subjects. The relationship update engine 412 (e.g., executed by or on behalf of each of the researchers) further can update the kinship relationship specified by relatedness data 416 in the dataset 402 based on the previous results, such as using a weighted voting strategy. For example, given a list of kinship relationship predictions from T iterations, k¹, k², . . . , k^{T}, a weighted kinship relationship k can be calculated as Σ₁^{T}w^{t}*k^{t}, where w^{t} is the weight associated with k^{t}, and

$${w}_{t} = \frac{\frac{1}{T + 1 - t}}{{\sum }_{t}^{T} \frac{1}{T + 1 - t}} .$$

The relationship update engine 412 can determine the majority weighted vote, k*=argmax k, and set the determined majority weighted vote as the kinship relationship between the pair of samples. The update module 400 can compute relatedness metrics (e.g., KING coefficients) and kinship relationships for each of the sample pairs.

Instead of outsourcing metadata all at once, the systems and methods described herein break down the metadata into smaller fractional subsets of data units (e.g., SNPs) and outsources each subset iteratively. The researchers can then combine the results from multiple iterations to improve the robustness of the relatedness-based approach and with increased privacy preservation compared to existing approaches. The resulting relatedness (e.g., kinship relationship) among samples can be used to remove or retain different samples based on the study that the researchers want to conduct (i.e., whether they need to remove related samples or keep only the family members). For example, the IDs of samples that should be filtered out can be provided to each researcher (e.g., by the server or determined locally by the synchronization module 112, 200), which can be used to provide respective filtered subsets of samples for such research. The researchers can then send data to the server for analyzing genetic information based on respective filtered subsets of samples provided by the researchers.

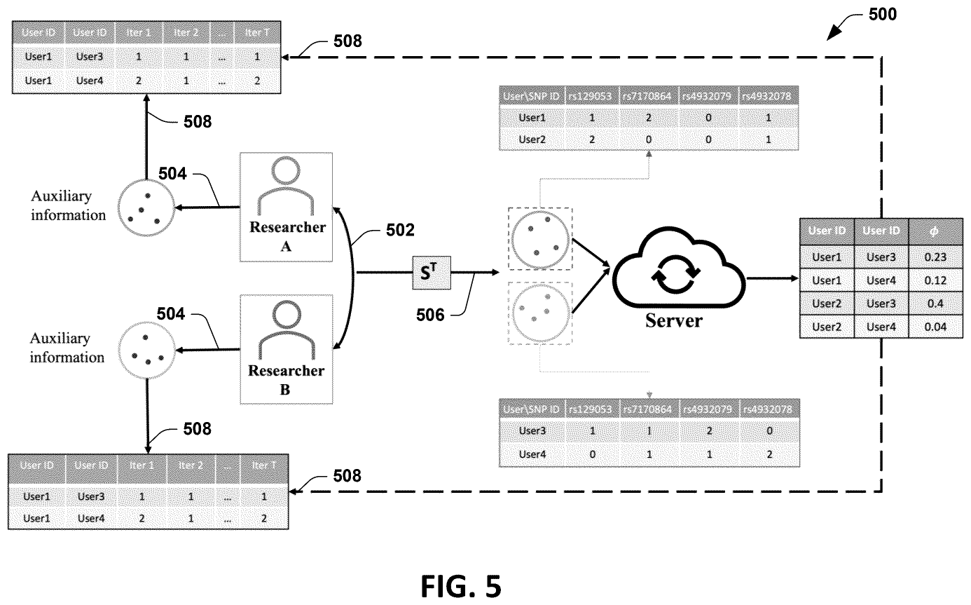

FIG. 5 depicts a workflow diagram 500 showing the overall framework that can be used to implement for an example privacy preserving system or method used by researchers for analyzing genomic data. As described herein, the systems and methods described herein are applicable to other data domains. In the example of FIG. 5, the workflow diagram 500 illustrates an iterative approach to determine relatedness between data samples that includes four stages: synchronization 502, initialization 504, outsourcing 506, and updating 508. For example, the synchronization stage 502 can include two or more researchers collaborating and initially deciding whether they will continue the process. Responsive to deciding to continue, the researchers agree on the subset of SNPs that is used to generate the metadata which is later sent to the server. The initialization stage 504 can include each researcher utilizing auxiliary information (e.g. simulated samples) and extracting the basic statistics, such as distributions of KING coefficients. The outsourcing stage 506 can include each researcher sending metadata to the server, which calculates and returns the pairwise KING coefficients to both researchers. The updating stage can then include each researcher locally updating the kinship relationships among sample pairs based on the pairwise KING coefficients received from the server.

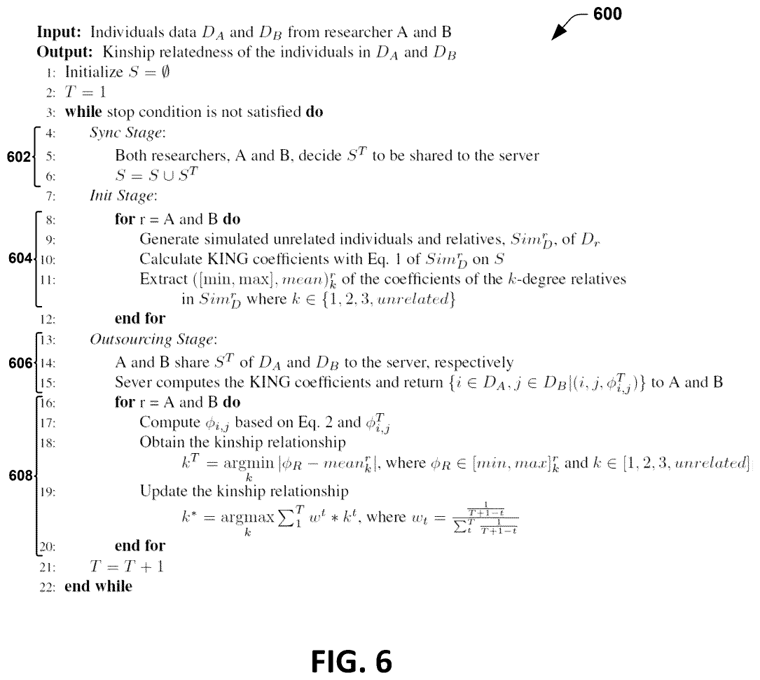

FIG. 6 depicts an example of pseudo code for an algorithm 600 that can be implemented in a privacy preserving system. The example pseudo code is shown for the domain of genomic research; however, the privacy preserving approach can be used in other domains and for different types of data. Similar to the example workflow of FIG. 5, the algorithm is an iterative process, in which each iteration can include a synchronization stage 602, an initialization stage 604, an outsourcing stage 606, and an updating stage 608. In the synchronization stage 602, the researchers mutually decide on the set of SNPs which are used to generate metadata (e.g., by synchronization module 112, 200). The selected set of SNPs (e.g., SNP IDs) is not revealed to the server as part of the metadata. In the initialization stage 604, each researcher can generate synthetic relatives and unrelated subjects (e.g., a simulated set) following Mendel's Laws. Each researcher can calculate (e.g., by initialization module 114, 300) KING coefficients on the simulated set and extracts the aggregate statistics for each kinship relationship group, such as ([min, max], mean)ₖ.

In the outsourcing stage 606, each researcher sends the metadata to the server. Since the metadata (e.g., metadata from other computers 132) does not contain the SNP IDs, the server cannot simply construct the SNP sequence of the samples that are included in the metadata. The server (e.g., incremental pairwise relatedness calculator 130) calculates the KING coefficients (e.g., incremental pairwise relatedness data 134, 406) based on the pooled metadata and returns each pair's coefficients to each of the researchers. The updating stage 608 can include each researcher calculating (e.g., by update module 118, 400) the KING coefficients on all the shared SNPs (see, e.g., Eq 2). Then, the results can be compared with the aggregate statistics, and each researcher determines the kinship relationship for each pair of subjects. Finally, each researcher updates the kinship relationship in the researcher's dataset based on the previous results, such as using a weighted voting strategy described herein. Each researcher can then take the majority weighted vote and set it as the kinship relationship between the samples. In the synchronization phase of the next iteration, the researchers can also decide if they want to continue. For example, if the kinship relationships for all the subjects remain the same, both researchers choose to end the iteration.

Determining the kinship relationships based on some relatedness measures that quantify the genetic information shared by subjects is a straightforward and efficient approach. However, the accuracy of these estimators is not guaranteed. The performance of these measures can be affected by the number of genetic markers, allele frequency distribution, and the kin structure of the population. A large set of SNPs can help to increase the accuracy and robustness of the relatedness measure-based estimators (e.g., KING). For example, the distributions of the KING coefficient for different kinship categories (i.e., relationship degrees) can be based on 20K SNPs. As described herein, however, using a large set of SNPs is not applicable in the federated setting for two reasons: (a) a large number of overlapping SNPs may not be feasible for all the participating parties; and (b) sharing a large number of SNPs can significantly increase the privacy risks.

In some examples, the systems and methods described herein (see, e.g., FIGS. 1-6 and 8) further can use auxiliary information, such as simulated distributions of KING coefficients and publicly available genomic datasets which contain real relatives, to determine (e.g., by simulated relatedness calculator 302) the cutoff values (e.g., thresholds) while classifying KING coefficients as belonging to these kinship categories.

By way of example, each researcher r creates a simulated set, denoted as Sim^{r}, that contains artificially generated relatives (up to third-degree relatives) and unrelated subjects of the subjects in their dataset. Given a set of SNPs and a pair of real subjects (i, j), the researcher initially calculates the KING coefficients of the simulated kinship categories (k) and obtains the descriptive statistics, i.e., {minimal, maximal, mean}ₖ, of the corresponding coefficients distributions. Then, the researcher r UPDATE MODULE classifies the kinship relationship of the pair of real subjects based on the KING coefficients (ϕ_{i,j}) and the distance to the simulated distributions. If the ϕ_{i,j}∈[min, max]ₜ, it is classified as a k-th degree kinship category. If the coefficient falls into multiple categories, the kinship relation is determined by the distance to the mean, and the closest category is chosen, i.e., k=argmin|ϕ_{R}−meanₖ| where ϕ_{R}∈[min, max]ₖk and k∈[1, 2, 3, unrelated].

As a further example, systems and methods described herein (see, e.g., FIGS. 1-6 and 8) can be adapted to utilize an informative score (IS) to help select subsets of data units for one or more iterations. For example, given SNP sequences from two subjects, the kinship relatedness based on the KING coefficient is determined by the pairwise combinations of SNPs (the total number of SNPs in which subjects are heterozygous, homozygous dominant, and homozygous recessive). When sharing of the whole SNP sequence is infeasible (as in the federated data examples described herein), the particular SNPs that are selected to compute the KING coefficients can be a factor to help achieve high accuracy in an efficient way. For instance, outsourcing a set of SNPs that have small impact on the KING coefficient (i.e., SNPs for which both subjects are homozygous recessive or homozygous dominant) is undesirable. Accordingly, the systems and methods herein can utilize an informative score (IS) to measure the impact of each SNP on KING coefficients calculation (or to measure the impact of other types of data units on the relatedness metrics). In the federated setting, the IS of each SNP is not directly available since the subjects are separated in two different datasets. Therefore, the simulated dataset, as described herein, can be used to calculate the informative score for each SNP. As an example, the informative score (IS) of for a respective SNP h can be defined as follows:

$$IS ( h ) = \frac{1}{\vert σ \vert} {\sum }_{( i , i ) ∈ σ} \vert log \frac{{ϕ}_{i , j}^{S}}{{ϕ}_{i , j}^{{S}_{- h}}} \vert \qquad ( Eq . 4 )$$

where:
σ denotes all the possible combinations of samples in the simulation set, and
S₋ₕ denotes the SNPs set without h (i.e., S₋ₕ=S\{h}).

During the iteration process, researchers first outsource the SNPs with high informative scores. When a set of SNPs have the same informative scores, a random or pseudorandom selection can be applied to break ties.

As mentioned above, the systems and methods described herein can help limit the privacy risk due to sharing metadata iteratively. In some examples, the server can be able to infer the sensitive information from the metadata if (a) at each iteration, the server can match the unordered SNP sequence shared by the researchers with a target's SNP sequence (i.e., a target subject, whose membership to a research dataset may be sensitive) and (b) the server is able to find the linkage among the SNPs shared across the iteration process.

To reduce the privacy risk, the systems and methods described herein can include two mitigation techniques. On the one hand, researchers select SNPs from different chromosomes for each iteration to remove the linkage among SNPs in different iterations. Also, or alternatively, researchers can remove the user IDs and shuffle the samples in each iteration. On the other hand, during each iteration, each researcher generates several synthetic SNPs and shares them as part of the metadata with the server. With carefully tuned parameters, the synthetic SNPs can be indistinguishable from real SNPs. As an example, to remove the noise introduced by the synthetic SNPs, researchers initially create two different sets of synthetic SNPs, named S^{F}1 and S^{F}2, then share 1 three batches of metadata, S_{F}∪S^{R}, S^{F}2∪S^{R}, and S^{F}1∪S^{F}2 respectively, with the server and obtain the KING coefficients, ϕ^{F}1 R, ϕ^{F}2 R, and ϕ^{F}1 F2. Based on Eq. 2, each researcher can recover the KING coefficients calculated on the real SNPs set R, ϕ^{R}, as follows:

$${ϕ}^{R} = \frac{{n}_{1 *}^{{F}_{1} R} {ϕ}^{{F}_{1} R} + {n}_{1 *}^{{F}_{2} R} {ϕ}^{{F}_{2} R} - {n}_{1 *}^{{F}_{1} {F}_{2}} {ϕ}^{{F}_{1} {F}_{2}}}{2 {n}_{1 *}^{R}} \qquad ( Eq . 5 )$$

As described herein, each researcher generates the synthetic SNPs independently.

As described herein, the systems and methods described herein (see, e.g., FIGS. 1-6 and 8) can employ three components to help preserve privacy and increase accuracy of relatedness metrics. Some examples of components and combinations thereof that can be implemented by the systems and methods are as follows (see, e.g., Table 1):
1. Increment: Iteratively outsource the SNPs and update the kinship relationships based on the predefined thresholds;
2. Increment+Sim: Iteratively outsource the SNPs and update the kinship relationships based on the simulated thresholds;
3. Increment+Informative: Iteratively outsource the SNPs based on the informative scores, and update the kinship relationships based on the predefined thresholds; and
4. Increment+Inform+Sim: Iteratively outsource the SNPs based on the informative scores, and update the kinship relationships based on the simulated thresholds

TABLE 1
Summary of Components combinations

| | Increment | | Sim | | Inform | | All | |
|---|---|---|---|---|---|---|---|---|
| | outsource update | | outsource update | | outsource update | | outsource update | |
| Increment | ★ | ★ | † | † | • | ★ | • | † |
| Sim | † | † | — | — | — | — | | |
| Inform | • | ★ | — | — | — | — | | |

Legend: Predefined threshold (★);
Simulated threshold (†);
Informative scores (•);
Not applicable (—)

The example methods can be compared with two baselines: centralized approach and privacy-preserving kinship identification (PPKI). The centralized approach assumes all the data is stored in one local node, and the computation is carried out locally. PPKI assumes there are two researchers and utilize an o̧-LDP variant for preserving privacy. o̧ is the privacy parameter which controls the trade-off between privacy and accuracy. Following the implementation of the PPKI, pick the parameters that result in high utility can be chosen, and therefore, we set o̧ to be 5.

As a further example, to evaluate the proposed components used in the systems and methods described herein, real genomic data from OpenSNP can be used along with randomly sampling a subset that contains about 200 unrelated subjects and about 3000 SNPs. Following Mendel's law, 200 pairs of synthetic relatives can be generated for each degree of kinship relationship (e.g., up to third-degree) of the subjects in the subset.

The performance of the proposed systems and methods in comparison with the two baselines is evaluated in terms of utility (e.g., measured by the correctness of identifying kinship relationships) and the relative privacy loss (e.g., measured by the number of SNPs required to achieve a certain correctness level). The approach that achieves higher kinship identification correctness and lower relative privacy loss can indicate a preferred approach. In an example, accuracy rate and Area Under the Receiver Operating Characteristic Curve (AUROC) scores can be used to measure the correctness of kinship relationships identification. Other accuracy metrics can be used in other examples.

As described herein, the number of outsourced SNPs can be used to measure the potential privacy risk in the federated setting. Denote the number of SNPs required by baseline q as |S|^{q} and the kinship identification correctness achieved by this baseline with |S|^{q} SNPs as c-correctness. The relative privacy loss (RPL(c)) of approach p to the baseline q can be defined in Eq. 5 as follows:

$${RPL ( c )}_{p , q} = \frac{{\vert S \vert}^{p} - {\vert S \vert}^{q}}{{\vert S \vert}^{q}} \qquad ( Eq . 5 )$$

where |S|^{p} is the minimal number of SNPs required by q to achieve c-correctness. In the following examples, the centralized method, in which all the data is stored in a single node and no privacy risk introduced, can be used as the baseline q. As per the above definition, a negative value is always preferred since it means a privacy gain compared to the centralized approach.

An example of the utility of identifying relatives is shown in Table 2. In an example, the size of the metadata (the number of SNPs) in each iteration can be fixed at 100 and continue the iteration until all the SNPs are shared with the server. In other examples, other numbers of data units can be used, which can be the same or different at each iteration. For the two baselines, the KING coefficients can be calculated using all the SNPs. From Table 2, it is shown that under task (a), all the proposed approaches outperform PPKI. Simulation set has a more significant impact than the other two components. Increment+Sim and Increment+Inform+Sim achieve higher accuracy rates and AUPRC scores compared to the other methods. Under task (b), we notice that Simulation set has a minor contribution. This is due to the low expressibility of the basic statistics including minimum, maximum, and mean. Samples containing different kinship categories can lead to a complicated KING coefficient distribution, which basic statistics cannot capture. To address this issue, the systems and methods described herein can incorporate the misclassification rate to determine the cutoff values (e.g., thresholds). However, the utility can still be improved solely by the Incremental Process and the weighted voting strategy described herein.

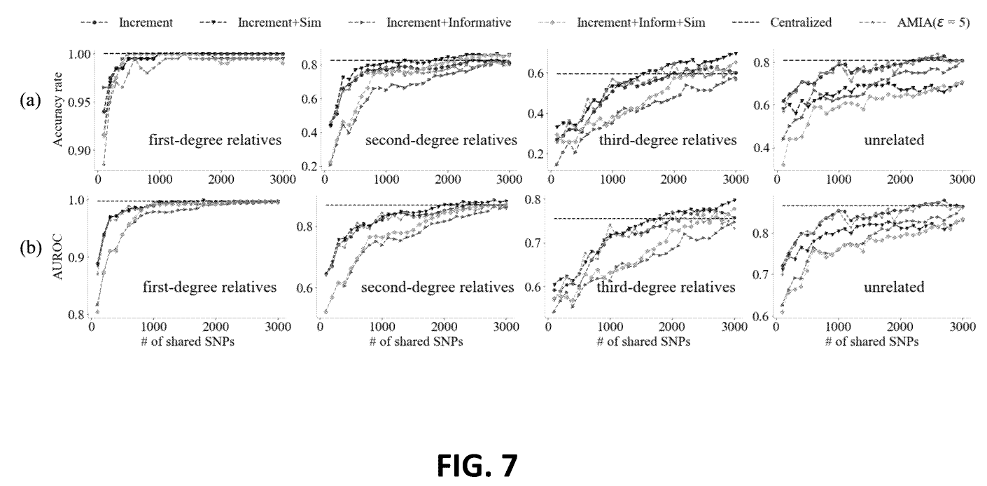

FIG. 7 depicts examples of graphs plotting accuracy for different degrees of relatedness as a function of the number of shared data units, which can be determined by systems and methods described herein (see, e.g., FIGS. 1-6 and 8). FIG. 7 also shows the change in utility for all the kinship relationships with different numbers of SNPs. The correctness of identifying kinship relatedness increases when more SNPs are provided. However, to identify higher degree kinship relationships, more SNPs are usually required. While no single approach (e.g., combination of components) dominates the rest, Incremental process and Simulation set show a greater improvement in terms of utility.

TABLE 2
Kinship identification correctness. The best performance is marked
in light blue font, and the second best is marked in blue font

| Methods | Task Accuracy | (a) AUROC | Task Accuracy | (b) AUROC |
|---|---|---|---|---|
| Increment | 0.803 | 0.870 | 0.908 | 0.868 |
| Increment + Sim | 0.821 | 0.881 | 0.814 | 0.858 |
| Increment + Informative | 0.800 | 0.869 | 0.877 | 0.808 |
| Increment + Inform + Sim | 0.816 | 0.877 | 0.900 | 0.800 |
| Centralized | 0.807 | 0.873 | 0.906 | 0.866 |
| PPKI [13] (ǫ = 5) | 0.799 | 0.866 | 0.903 | 0.866 |

Based on the accuracy rate and AUPRC scores of the centralized setting (which is considered as the main baseline in this experiment), the relative privacy loss of the proposed methods and PPKI can be computed, such as shown in Table 3. In Table 3, “>0” suggests that the relative privacy loss is undefined since the number of the required SNPs is larger than the dataset size. From Table 3, the Increment+Sim components are shown to achieve the best and the second best RPL results in terms of AUROC for tasks (a) and (b), respectively. The obtained results show that Increment+Sim require approximately 24% fewer SNPs in order to achieve the same kinship identification correctness as the baseline.

TABLE 3
Relative privacy loss.

| Methods | Task (a) RPL(acc) RPL(auc) | | Task (b) RPL(acc) RPL(auc) | |
|---|---|---|---|---|
| Increment | >0 | >0 | −0.19 | −0.09 |
| Increment + Sim | −0.25 | −0.24 | >0 | −0.09 |
| Increment + Informative | >0 | >0 | >0 | >0 |
| Increment + Inform + Sim | −0.03 | >0 | >0 | >0 |
| PPKI [13] (ǫ = 5) | −0.05 | >0 | −0.24 | −0.19 |

The proposed framework assumes that the researchers need to synchronize and maintain the samples' kinship relatedness once the server returns the results at each iteration. Hence, the computation and communication overhead increases linearly with the number of iterations. Researchers can stop the iteration process early if the goal is to identify the lower degrees of relatives, such as shown in FIG. 7 (e.g., only a small number of SNPs, 200-400, enables them to achieve a high accuracy rate of identifying first-degree relatives). In order to identify higher-degree relatives and reduce the computation and communication overhead, researchers can (e.g., through synchronization module 112, 200) increase the number of shared SNPs (m) at each iteration. In the example of Table 4, a change in accuracy is shown when considering different numbers (m) of shared SNPs in an iteration. From Table 4, the communication and computation overhead can be reduced drastically with a low impact on the utility. For instance, when m increases from 100 to 600, the number of iterations can decrease from 30 to 5, and the AUPRC score of Increment+Sim slightly decreases from 0.881 to 0.879 for task (a).

TABLE 4
Utility with varying numbers of shared SNPs in each
iteration. Results are based on 3000 SNPs in total

| | | m = | Task | (a) | m = | m = | Task (b) m = | | |
|---|---|---|---|---|---|---|---|---|---|
| | Methods | 100 | m = 200 | m = 400 | 600 | 100 | 200 m = 400 | | m = 600 |
| | Increment | 0.806 | 0.805 | 0.800 | 0.802 | 0.912 | 0.911 | 0.910 | 0.908 |
| Accuracy | Increment + Sim | 0.823 | 0.822 | 0.817 | 0.819 | 0.832 | 0.829 | 0.828 | 0.825 |
| | Increment + | 0.800 | 0.791 | 0.787 | 0.790 | 0.879 | 0.884 | 0.886 | 0.889 |
| | Informative | | | | | | | | |
| | Increment + | 0.816 | 0.813 | 0.803 | 0.804 | 0.900 | 0.899 | 0.893 | 0.899 |
| | Inform + Sim | | | | | | | | |
| | Increment | 0.870 | 0.870 | 0.867 | 0.869 | 0.868 | 0.866 | 0.863 | 0.861 |
| AUROC | Increment + Sim | 0.881 | 0.881 | 0.877 | 0.879 | 0.858 | 0.858 | 0.858 | 0.858 |
| | Increment + | 0.869 | 0.864 | 0.860 | 0.862 | 0.808 | 0.820 | 0.833 | 0.848 |
| | Informative | | | | | | | | |
| | Increment + | 0.877 | 0.876 | 0.869 | 0.870 | 0.800 | 0.798 | 0.785 | 0.803 |
| | Inform + Sim | | | | | | | | |

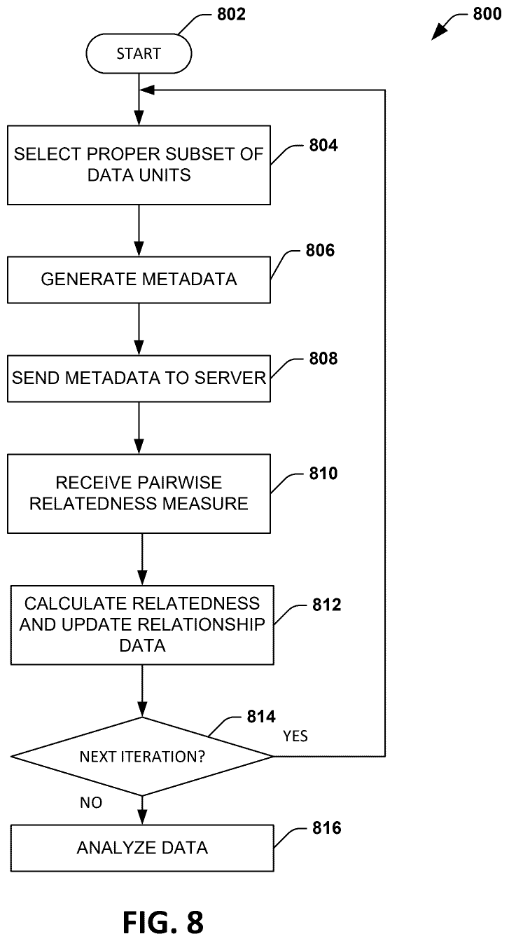

FIG. 8 is a flow diagram 800 depicting an example method to preserve privacy for collaborative data analysis. While, for purposes of simplicity of explanation, the method 800 of FIG. 8 is shown and described as executing serially, it is to be understood and appreciated that such methods are not limited by the illustrated order, as some aspects could, in other examples, occur in different orders and/or concurrently with other aspects from that disclosed herein. Moreover, not all illustrated features may be required to implement a method. The methods or portions thereof can be implemented as instructions stored in one or more non-transitory machine readable media and be executed by a processor of one or more computer devices, for example, to cause the processor to perform the method. The method 800 can be implemented according to the systems and algorithm described herein. Accordingly, the description of FIG. 8 can refer to certain aspects of FIGS. 1-6. For sake of simplicity, the method 800 is described from the perspective of a given user (e.g., researcher) and a corresponding instance of the method can be implemented in parallel by each other user with which the given user is collaborating.

The method 800 begins at 802, which can include initiating collaboration between two or more users (e.g., researchers). This can include authorizing such collaboration within a federated system (e.g., managed by server 106), such as responsive to user inputs by the collaborating users. Communication between such users can be encrypted and/or occur through a secure channel, as described herein.

At 804, the method includes selecting a proper subset of data units in each of a plurality of samples stored in a dataset. As described herein, each sample of the plurality of samples stored in the dataset includes a respective plurality of data units. In some examples, the subset of data units can include SNPs. An information score can also be computed and the selection of data units at 804 can be based on the informative score. For example, the selection of the subset of data units at 804 can be implemented by synchronization module 112, 200.

At 806, metadata is generated based on the subset of data units selected at 804. In some examples, the auxiliary information, which can include providing simulated (e.g., artificial) samples from which statistical data can be extracted. For example, the generation of metadata at 806 can be implemented as part of an initialization stage by initialization module 114, 300.

At 808, the method includes sending the metadata (e.g., from the given user's computer) to a second computer (e.g., server) through a communications link. At 810, the method includes receiving (e.g., at the given user's computer from the server) a pairwise measure of relatedness. The pairwise measure of relatedness is based on the metadata from the given user's computer and other metadata from at least one other computer of a user with which the give user is collaborating. The measure of relatedness quantifies a similarity between samples based on the proper subset of data units generated from the given user's dataset and the proper subset of data units generated for at least one other dataset. For example, the sending and receiving at 808 and 810 can be implemented as part of an outsourcing stage by outsourcing module 114, 300.

At 812, the method 800 includes updating the plurality of samples stored in the dataset based on the pairwise measure of relatedness received at 810. The updating at 812 further can be based on the measure of relatedness for the current iteration as well as one or more prior iterations, as described herein. For example, the updating at 812 can be implemented by the update module 118, 400 and thus can include calculating a pairwise measure of relatedness and classifying samples into categories of relatedness (e.g., degrees of kinship).

At 814, a determination is made as to whether a next iteration is to occur. Responsive to determining that a next iteration is to occur (YES), the method returns to perform a next iteration and repeat each of the selecting, generating, sending, receiving, and updating for another iteration at 804-812. The metadata sent at 808 can be unlinked to metadata sent at other iterations to enhance the privacy preserving function of the method 800. Responsive to determining that a next iteration is not to occur (NO), the method proceeds to 816. At 816, data can be analyzed based on the updated relatedness between datasets. For example, the samples in the respective datasets being analyzed at 816 can be filtered by one of removing or retaining a subset of samples from the data samples stored in dataset to provide a first filtered subset of the data samples based on the updated measure of relatedness for the respective samples (at 812). The analysis of the data samples can include analyzing genetic information represented in the filtered subsets of the samples (e.g., samples from the respective federated datasets).

In view of the foregoing, it is to be understood that by sharing a limited number of SNPs or other data units the systems and methods described herein achieve a good accuracy in estimating the degree of relatedness between samples. This allows the systems and methods described herein to estimate kinship relationships while preserving privacy by limiting the amount of information shared. Moreover, the flexibility of combining various features described herein can facilitate processing heterogeneous datasets. Collaborators can choose the best performing combination of these components according to their required kinship estimation accuracy. The systems and methods described herein thus can be lightweight, easy to implement on the researcher's side, and does not require a completely trusted server. In some examples, the systems and methods described herein can implement secure multi-party computation (secure-MPC) protocols, such as ABY3, Falcon, Function Secret Sharing (FSS), SPDZ, etc.

### Example Embodiments

Several aspects of the present technology are set forth in the following numbered examples.

Example 1. A method comprising:
generating, on or by a first computer, first metadata representative of a first proper subset of data units in each of a plurality of samples stored in a first dataset, in which each sample of the plurality of samples stored in the first dataset includes a plurality of data units;
generating, on or by a second computer, second metadata representative of a second proper subset of data units in each of a plurality of samples stored in a second dataset, in which each sample of the plurality of samples stored in the second dataset includes a plurality of data units;
sending the first metadata to a third computer through a first communications link;
sending the second metadata to the third computer through a second communications link;
calculating, by the third computer, a measure of relatedness between the samples stored in the first dataset and the samples stored in the second dataset based on the first metadata and the second metadata; and
sending the measure of relatedness to each of the first computer and the second computer.
   Example 2. The method of example 1, further comprising:
updating, by the first computer, the plurality of samples stored in the first dataset based on the measure of relatedness; and
updating, by the second computer, the plurality of samples stored in the second dataset based on the measure of relatedness.
   Example 3. The method of example 2, wherein the updated plurality of samples stored in the first dataset includes data representing the relatedness of at least some of the proper subset of data units in each of the samples stored in the first dataset with respect to corresponding samples stored in the second dataset, and
wherein the updated plurality of samples stored in the second dataset includes data representing the relatedness of at least some of the proper subset of data units in each of the samples stored in the second dataset with respect to corresponding samples stored in the first dataset.
   Example 4. The method of example 1 or 2, wherein each of the data units includes a single nucleotide polymorphism (SNP) of a multitude of SNPs in each of the samples,
wherein the measure of relatedness includes coefficients representing a kinship relationship between each pair of the SNPs represented by the first and second metadata, and
wherein the updating for each of first and second computers further comprises:
   computing a value for the relatedness based on the coefficient for each pair of the SNPs for at least two iterations of the method; and
   classifying a degree of the kinship relationship based on the computed value for each pair of the SNPs based on one or more thresholds.
      Example 5. The method of any preceding example, wherein the first metadata and the second metadata include identifiers for a common proper subset of respective data units for the samples stored in the first and second datasets.
      Example 6. The method of example 5, wherein the identifiers are encoded identifiers, in which each of encoded identifiers represents a respective data unit.
      Example 7. The method of example 6, wherein each of the data units represents a genetic marker, and each of the encoded identifiers represents a respective genetic marker.
      Example 8. The method of example 7, wherein each of the data units represents a single nucleotide polymorphism (SNP), and each of encoded identifiers represents a respective SNP.
      Example 9. The method of any preceding example, further comprising:
generating, on or by the first computer, third metadata representative of a third proper subset of data units in each of the plurality of samples stored in the first dataset;
generating, on or by the second computer, fourth metadata representative of a fourth proper subset of data units in each of the plurality of samples stored in the second dataset;
sending the third metadata to the third computer;
sending the fourth metadata to the third computer;
calculating, by the third computer, a measure of relatedness between the samples stored in the first dataset and the samples stored in the second dataset based on the third metadata and the fourth metadata; and
sending the measure of relatedness to each of the first computer and the second computer.
   Example 10. The method of example 9, wherein each of the first and second proper subsets includes a first number of data units, and
wherein each of the third and fourth proper subsets includes a second number of data units.
   Example 11. The method of example 10, wherein the first number of data units and the second number of data units one of the same or different.
   Example 12. The method of any preceding example, further comprising:
one of removing or retaining a subset of samples from the samples stored in the first dataset to provide a first filtered subset of the samples based on the measure of relatedness;
one of removing or retaining a subset of samples from the samples stored in the second dataset to provide a second filtered subset of the samples based on the measure of relatedness; and
analyzing genetic information represented in the first filtered subset of the samples and the second filtered subset of the samples.
   Example 13. A method comprising:
selecting a proper subset of data units in each of a plurality of samples stored in a first dataset, in which each sample of the plurality of samples stored in the first dataset includes a respective plurality of data units;
generating metadata based on the selected subset of data units;
sending the metadata to a second computer through a communications link;
receiving a measure of relatedness determined by the second computer, in which the measure of relatedness is based on the generated metadata and other metadata from at least one other computer, and the measure of relatedness quantifies a similarity between samples based on the proper subset of data units for samples stored in the first dataset and a proper subset of data units for samples stored in at least one other dataset; and
updating the first dataset based on the received measure of relatedness, in which each of the selecting, generating, sending, receiving and updating is repeated for a plurality of iterations, and the updating performed for at least one iteration is based on measures of relatedness for a current iteration and one or more preceding iterations.
   Example 14. The method of example 13, wherein each of the data units defines a single nucleotide polymorphism (SNP) of a multitude of SNPs,
wherein the received measure of relatedness includes coefficients representing a kinship relationship between each pair of the SNPs represented by the generated metadata and the other metadata from the at least one other computer, and
wherein the updating further comprises:
   computing a value for the relatedness based on the coefficient for each pair of the SNPs for at least two of the iterations; and
   classifying a degree of the kinship relationship based on the computed value for each pair of the SNPs based on one or more thresholds.
      Example 15. The method of example 13 or 14, wherein the generated metadata and the other metadata from at least one other computer include identifiers for a common proper subset of respective data units for the samples stored in the first dataset and at least one other dataset, and
wherein, for each iteration, a number of data units for the generated metadata and other metadata is one of the same or different than another iteration.
   Example 16. The method of example 15, wherein the identifiers are encoded identifiers, in which each of encoded identifiers represents a respective data unit.
   Example 17. The method of any of examples 13-16, wherein each of the data units represents a single nucleotide polymorphism (SNP), and each of encoded identifiers represents a respective SNP.
   Example 18. The method of any of examples 13-17, further comprising:
one of removing or retaining a subset of samples from the samples stored in the first dataset to provide a filtered subset of the samples stored in the first dataset based on the updated dataset; and
analyzing genetic information based on the filtered subset of the samples.
   Example 19. A system, comprising:
non-transitory memory to store instructions and data, in which the data comprises a first dataset that includes a plurality of samples, each sample including a plurality of data units; and
one or more processors coupled to the memory, in which the instructions are executable by the one or more processors, the instructions comprising:
synchronization code to select a proper subset of the data units in each of a plurality of samples stored in the first dataset, in which each sample of the plurality of samples stored in the first dataset includes a respective plurality of data units;
metadata generator code to generate metadata based on the selected subset of data units;
outsource code to send the generated metadata to a server computer through a communications link, the outsource code also to receive a measure of relatedness from the server computer, in which the received measure of relatedness is based on the generated metadata and other metadata from at least one other computer, and the measure of relatedness quantifies a similarity between samples based on the proper subset of data units for samples stored in the first dataset and a proper subset of data units for samples stored in at least one other dataset; and
update code to update the first dataset based on the received measure of relatedness, in which each of the synchronization code, the metadata generator code, the outsource code, and the update code is executed for each of a plurality of iterations, and the updating at each of the iterations is based on measures of relatedness for a current iteration and one or more prior iterations.
   Example 20. The system of example 19, wherein each of the data units defines a single nucleotide polymorphism (SNP) of a multitude of SNPs stored for each of the samples in the first dataset,
wherein the received measure of relatedness includes coefficients representing a kinship relationship between samples based on each pair of the SNPs represented by the generated metadata and the other metadata from the at least one other computer, and
wherein the update code is further programmed to:
   compute a value for the relatedness based on the coefficient for each pair of the SNPs for at least two of the iterations; and
   classify a degree of the kinship relationship based on the computed value for each pair of the SNPs based on one or more thresholds.
      Example 21. A method can include generating first metadata representative of a first proper subset of data units in each of a plurality of samples stored in a first dataset, in which each sample of the plurality of samples stored in the first dataset includes a plurality of data units. The method can also include generating second metadata representative of a second proper subset of data units in each of a plurality of samples stored in a second dataset, in which each sample of the plurality of samples stored in the second dataset includes a plurality of data units. The method can also include sending the first and second metadata to a computer. The method can also include calculating a measure of relatedness between the samples stored in the first dataset and the samples stored in the second dataset based on the first metadata and the second metadata.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a computer device.

Also, certain examples have been described herein with reference to block illustrations of methods, systems, and computer program products. It will be understood that blocks of the illustrations, and combinations of blocks in the illustrations, can be implemented by computer-executable instructions. These computer-executable instructions may be provided to one or more processors of a general purpose computer, special purpose computer, or other programmable data processing apparatus (or a combination of devices and circuits) to produce a machine, such that the instructions, which execute via the processor, cause the processor to implement the functions specified in the block or blocks.

These computer-executable instructions may also be stored in computer-readable memory that can direct a computer or other programmable data processing apparatus to function in a particular manner, such that the instructions stored in the computer-readable memory result in an article of manufacture including instructions which implement the function specified in the flowchart block or blocks. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart block or blocks.

Where the disclosure or claims recite “a,”, “an,” “a first,” or “another” element, or the equivalent thereof, it should be interpreted to include one or more than one such element, neither requiring nor excluding two or more such elements. As used herein, the term “based on” means based at least in part on. As used herein, the term “and/or” can include any and all combinations of one or more of the associated listed items.

As used herein, the term “includes” means includes but not limited to, the term “including” means including but not limited to what is listed. The term “based on” means based at least in part on. Additionally, where the disclosure or claims recite “a,” “an,” “a first,” or “another” element, or the equivalent thereof, it should be interpreted to include one or more than one such element, neither requiring nor excluding two or more such elements.

What have been described above are examples of the disclosure. It is, of course, not possible to describe every conceivable combination of components or method for purposes of describing the disclosure, but one of ordinary skill in the art will recognize that many further combinations and permutations of the disclosure are possible. Accordingly, the disclosure is intended to embrace all such alterations, modifications, and variations that fall within the scope of this application, including the appended claims.

All references, publications, and patents cited in the present application are herein incorporated by reference in their entirety.

Having described the invention, we claim:

1. A method comprising:
generating, on or by a first computer, first metadata representative of a first proper subset of data units in each of a plurality of samples stored in a first dataset, in which each sample of the plurality of samples stored in the first dataset includes a plurality of data units;
generating, on or by a second computer, second metadata representative of a second proper subset of data units in each of a plurality of samples stored in a second dataset, in which each sample of the plurality of samples stored in the second dataset includes a plurality of data units;
sending the first metadata to a third computer through a first communications link;
sending the second metadata to the third computer through a second communications link;
calculating, by the third computer, a measure of relatedness between the samples stored in the first dataset and the samples stored in the second dataset based on the first metadata and the second metadata; and
sending the measure of relatedness to each of the first computer and the second computer.

2. The method of claim 1, further comprising:
updating, by the first computer, the plurality of samples stored in the first dataset based on the measure of relatedness; and
updating, by the second computer, the plurality of samples stored in the second dataset based on the measure of relatedness.

3. The method of claim 2, wherein the updated plurality of samples stored in the first dataset includes data representing the relatedness of at least some of the proper subset of data units in each of the samples stored in the first dataset with respect to corresponding samples stored in the second dataset, and
wherein the updated plurality of samples stored in the second dataset includes data representing the relatedness of at least some of the proper subset of data units in each of the samples stored in the second dataset with respect to corresponding samples stored in the first dataset.

4. The method of claim 2, wherein each of the data units includes a single nucleotide polymorphism (SNP) of a multitude of SNPs in each of the samples,
wherein the measure of relatedness includes coefficients representing a kinship relationship between each pair of the SNPs represented by the first and second metadata, and
wherein the updating for each of first and second computers further comprises:
computing a value for the relatedness based on the coefficient for each pair of the SNPs for at least two iterations of the method; and
classifying a degree of the kinship relationship based on the computed value for each pair of the SNPs based on one or more thresholds.

5. The method of claim 1, wherein the first metadata and the second metadata include identifiers for a common proper subset of respective data units for the samples stored in the first and second datasets.

6. The method of claim 5, wherein the identifiers are encoded identifiers, in which each of encoded identifiers represents a respective data unit.

7. The method of claim 6, wherein each of the data units represents a genetic marker, and each of the encoded identifiers represents a respective genetic marker.

8. The method of claim 7, wherein each of the data units represents a single nucleotide polymorphism (SNP), and each of encoded identifiers represents a respective SNP.

9. The method of claim 1, further comprising:
generating, on or by the first computer, third metadata representative of a third proper subset of data units in each of the plurality of samples stored in the first dataset;
generating, on or by the second computer, fourth metadata representative of a fourth proper subset of data units in each of the plurality of samples stored in the second dataset;
sending the third metadata to the third computer;
sending the fourth metadata to the third computer;
calculating, by the third computer, a measure of relatedness between the samples stored in the first dataset and the samples stored in the second dataset based on the third metadata and the fourth metadata; and
sending the measure of relatedness to each of the first computer and the second computer.

10. The method of claim 9, wherein each of the first and second proper subsets includes a first number of data units, and
wherein each of the third and fourth proper subsets includes a second number of data units.

11. The method of claim 10, wherein the first number of data units and the second number of data units one of the same or different.

12. The method of claim 1, further comprising:
one of removing or retaining a subset of samples from the samples stored in the first dataset to provide a first filtered subset of the samples based on the measure of relatedness;
one of removing or retaining a subset of samples from the samples stored in the second dataset to provide a second filtered subset of the samples based on the measure of relatedness; and
analyzing genetic information represented in the first filtered subset of the samples and the second filtered subset of the samples.

13. A method comprising:
selecting a proper subset of data units in each of a plurality of samples stored in a first dataset, in which each sample of the plurality of samples stored in the first dataset includes a respective plurality of data units;
generating metadata based on the selected subset of data units;
sending the metadata to a second computer through a communications link;
receiving a measure of relatedness determined by the second computer, in which the measure of relatedness is based on the generated metadata and other metadata from at least one other computer, and the measure of relatedness quantifies a similarity between samples based on the proper subset of data units for samples stored in the first dataset and a proper subset of data units for samples stored in at least one other dataset; and
updating the first dataset based on the received measure of relatedness, in which each of the selecting, generating, sending, receiving and updating is repeated for a plurality of iterations, and the updating performed for at least one iteration is based on measures of relatedness for a current iteration and one or more preceding iterations.

14. The method of claim 13, wherein each of the data units defines a single nucleotide polymorphism (SNP) of a multitude of SNPs,
wherein the received measure of relatedness includes coefficients representing a kinship relationship between each pair of the SNPs represented by the generated metadata and the other metadata from the at least one other computer, and
wherein the updating further comprises:
computing a value for the relatedness based on the coefficient for each pair of the SNPs for at least two of the iterations; and
classifying a degree of the kinship relationship based on the computed value for each pair of the SNPs based on one or more thresholds.

15. The method of claim 13, wherein the generated metadata and the other metadata from at least one other computer include identifiers for a common proper subset of respective data units for the samples stored in the first dataset and at least one other dataset, and
wherein, for each iteration, a number of data units for the generated metadata and other metadata is one of the same or different than another iteration.

16. The method of claim 15, wherein the identifiers are encoded identifiers, in which each of encoded identifiers represents a respective data unit.

17. The method of claim 16, wherein each of the data units represents a single nucleotide polymorphism (SNP), and each of encoded identifiers represents a respective SNP.

18. The method of claim 13, further comprising:
one of removing or retaining a subset of samples from the samples stored in the first dataset to provide a filtered subset of the samples stored in the first dataset based on the updated dataset; and
analyzing genetic information based on the filtered subset of the samples.

19. A system, comprising:
non-transitory memory to store instructions and data, in which the data comprises a first dataset that includes a plurality of samples, each sample including a plurality of data units; and
one or more processors coupled to the memory, in which the instructions are executable by the one or more processors, the instructions comprising:
synchronization code to select a proper subset of the data units in each of a plurality of samples stored in the first dataset, in which each sample of the plurality of samples stored in the first dataset includes a respective plurality of data units;
metadata generator code to generate metadata based on the selected subset of data units;
outsource code to send the generated metadata to a server computer through a communications link, the outsource code also to receive a measure of relatedness from the server computer, in which the received measure of relatedness is based on the generated metadata and other metadata from at least one other computer, and the measure of relatedness quantifies a similarity between samples based on the proper subset of data units for samples stored in the first dataset and a proper subset of data units for samples stored in at least one other dataset; and
update code to update the first dataset based on the received measure of relatedness, in which each of the synchronization code, the metadata generator code, the outsource code, and the update code is executed for each of a plurality of iterations, and the updating at each of the iterations is based on measures of relatedness for a current iteration and one or more prior iterations.

20. The system of claim 19, wherein each of the data units defines a single nucleotide polymorphism (SNP) of a multitude of SNPs stored for each of the samples in the first dataset,
wherein the received measure of relatedness includes coefficients representing a kinship relationship between samples based on each pair of the SNPs represented by the generated metadata and the other metadata from the at least one other computer, and
wherein the update code is further programmed to:
compute a value for the relatedness based on the coefficient for each pair of the SNPs for at least two of the iterations; and
classify a degree of the kinship relationship based on the computed value for each pair of the SNPs based on one or more thresholds.
